(19) United States
(12) Patent Application Publication
(10) Pub. No.: US 2026/0360052 A1
(43) Pub. Date: Nov. 12, 2026

(54) POINT CLOUD-BASED THREE-DIMENSIONAL OPTICAL COHERENCE TOMOGRAPHY VOLUMETRIC IMAGING

(71) Applicant: Northwestern University, Evanston, IL (US)

(72) Inventors: Raymond S. FANG, Evanston, IL (US); Hao ZHANG, Evanston, IL (US); Cheng SUN, Evanston, IL (US); Fengyuanshan XU, Evanston, IL (US)

(21) Appl. No.: 19/663,858

(22) Filed: Apr. 30, 2026

Related U.S. Application Data
(60) Provisional application No. 63/803,106, filed on May 9, 2025.

(51) Int. Cl. G06T 12/30; A61B 3/10; A61B 3/12; G06T 7/00; G06T 7/11; G06T 19/20

(52) U.S. Cl. CPC G06T 12/30; A61B 3/102; A61B 3/12; G06T 7/0012; G06T 7/11; G06T 19/20; G06T 2207/10028; G06T 2207/10101; G06T 2207/20021; G06T 2207/30041; G06T 2210/41; G06T 2210/56; G06T 2219/2004; G06T 2219/2016; G06T 2219/2021

(57) ABSTRACT

Imaging complex, non-planar anatomies with optical coherence tomography (OCT) is often limited by the optical field of view (FOV) achieved in a single volumetric acquisition. Systems and methods described herein provide for OCT beyond the FOV limit and for the construction of compound volumetric image representing a compound imaging volume (CIV) by montaging of multiple individual OCT volumetric images from adjacent volumetric images. Provided are processes for mapping a plurality of partially overlapping OCT volumetric images to obtain an expanded FOV by OCT, particularly for curved surfaces.

### CROSS-REFERENCE TO RELATED APPLICATIONS

This application claims the benefit of priority to U.S. Provisional Patent Application No. 63/803,106, filed May 9, 2025, which is hereby incorporated by reference to the extent not inconsistent herewith.

### STATEMENT REGARDING FEDERALLY SPONSORED RESEARCH OR DEVELOPMENT

This invention was made with government support under Award Numbers U01EY033001, R01EY034740, and F30EY034033 awarded by the National Institutes of Health. The government has certain rights in the invention.

### BACKGROUND

In the past three decades, optical coherence tomography (OCT) has revolutionized fundamental investigations in various disciplines, including cardiology, dermatology, and ophthalmology. Additionally, OCT has transformed the clinical management for nearly all blinding diseases. OCT is a non-invasive microscopic imaging modality that acquires volumetric data by detecting back-scattered photons at each optical illumination position and translating the optical focus of illumination to cover the region of interest (ROI). Such a data acquisition scheme has remained largely unchanged since OCT was first reported in 1991.

OCT typically has a micrometer-scale resolution of 3D volumes. However, OCT is fundamentally limited by a limited field of view (FOV). The FOV is limited to, at best, a few mm. This provides fundamental issues and obstacles for imaging periphery of curved surface, such as the retinal peripheral region. One way to address this limitation is to montage together many smaller FOV (see, e.g., WO 2024/226530), which is specifically incorporated by reference herein. Many conventional systems, however, do not perform this montaging in three dimensions (3D). Others have merged individual cross-sections together by maximizing cross-correlation of individual cross section. Others have designed lenses for wide field imaging, but that suffers from low scan density. Additionally, mirror artifacts are often present for single-volume acquisitions if the depth of acquisition, limited to a few mm, is too large. The issue with depth is particularly problematic for curved surfaces.

Provided herein is methodology for automatically montaging together 3D volumes of OCT data to image a large field of view in 3D. This has important implications for diagnosis of diseases involving peripheral retina, which is usually not imaged in conventional OCT methodologies.

### SUMMARY

OCT imaging technology has significantly advanced retinal care. However, commercial systems are limited by a relatively small FOV. This is addressed herein by increasing the FOV of OCT by volumetrically montaging multiple OCT datasets of the retina in three dimensions (3D). We performed volumetric montaging by representing the internal limiting membrane (ILM) of each volume as a point cloud and using the point clouds to compute the transformations mapping each volume to a common reference frame. Other retinal layers such as the retinal pigment epithelium (RPE) or any combination of layers (RPE+ILM) may also be used to generate the point cloud. Using the distance between point clouds, we find the error in montaging volumes is less than the lateral pixel size. Overall, the methodology provided herein for volumetric montaging has applications for better understanding the 3D structure of the retina across a larger FOV.

Provided herein are methods for expanding a field of view (FOV) in three dimensions of an object to be imaged by optical coherence tomography (OCT) by providing a plurality of OCT volumetric images comprising multiple overlapping pairs of OCT volumetric images. Each of the OCT volumetric images are represented as a point cloud corresponding to a surface of each of the OCT volumetric images. Common landmark point(s) between overlapping OCT volumetric images are identified. Each of the OCT volumetric images are mapped to a common reference frame. This mapping may be by: a rigid transformation including translation, rotation and reflection of the common landmark point(s) between each of the overlapping pairs of OCT volumetric images; refining the rigid transformation by recalculating the rigid transformation based on the point clouds of the overlapping region in addition to that of the landmark points; and a deformation registration to account for local deformations outside of the translation, rotation, and reflection of the common landmark point(s). A merged volume is formed from the mapped OCT volumetric images in the common reference frame; thereby expanding the FOV of the object.

The mapping may further comprise dividing the point cloud representing each of the OCT volumetric images into strips; calculating a deformation vector field from the strips by a strip-based algorithm; and using both the rigid transformation and the deformation vector field to map each of the OCT volumetric images to the common reference frame. The deformation vector field may improve alignment of the mapped OCT volumetric images that are otherwise subject to motion-based errors.

The plurality of OCT volumetric images may comprise actively obtaining the plurality of OCT volumetric images of the object or obtaining from a previously imaged object.

Any of the methods may further have a top surface of each of the OCT volumetric images that is an internal limiting membrane (ILM).

Any of the methods may further have a top surface of a subset of the plurality of OCT volumetric images that is a retinal anatomical layer after segmentation.

In an embodiment, the object to be imaged is the eye, and more particularly, a retina. An object that is a retina has a number of relevant applications. For example, the merged volume may be used to assess a surface topology parameter of the retina. In further examples, the merged volume from OCT of the retina can be used for one or more of: measuring a retinal curvature; diagnosing pathological myopia from retinal topology; characterizing a peripheral lesion (including 3D structure); point cloud-based eye tracking; or locating posterior staphylomas in a peripheral eye or retina region.

The methods are particularly relevant for increasing a FOV and axial depth without adversely impacting a lateral sampling density.

The expanding the FOV can be characterized by a lateral scanning dimension that is greater than or equal to 18 mm, including up to 30 mm, depending on the OCT hardware. In view of the average human eye diameter that is around 24 mm, a lateral scanning dimension may be greater than or equal to 18 mm and less than or equal to 30 mm, including between 20 mm and 28 mm.

The merged volume can be characterized as having an increased axial depth compared to the provided OCT volumetric images, including 1.5 to 2.5 times an axial depth of an individual OCT volumetric image, thereby minimizing risk of a mirror artifact.

The mean surface distance between adjacent point clouds after mapping may be less than a pixel lateral dimension.

The expanding the FOV may be quantitatively defined, including characterized by any one or more of: an increase in a lateral surface area between an individual OCT volumetric image lateral surface area (e.g., 30 mm² to 100 mm²) and the merged volume lateral surface area (100 mm² to 350 mm²), including a ratio of between 2.5 and 4.0; an increase in a volume between an individual OCT volumetric image volume (e.g., 70 mm³ to 175 mm³) and the merged volume (e.g., 250 mm³ to 1,100 mm³), including a ratio of between 3.5 and 7.

Also provided herein are systems for performing any of the methods described herein. For example, provided are systems for constructing an expanded FOV of an object to be imaged from a plurality of OCT volumetric images. The system may comprise: an OCT imaging system; and processing circuitry configured to: construct, based on data provided by the OCT imaging system, a plurality of OCT volumetric images; and perform the method according to any of the methods described herein.

Also provided herein are non-transitory, computer-readable medium having stored thereon processor executable instructions that, when executed by one or more processors, cause the one or more processors to perform any of the methods described herein.

Without wishing to be bound by any particular theory, there may be discussion herein of beliefs or understandings of underlying principles relating to the devices and methods disclosed herein. It is recognized that regardless of the ultimate correctness of any mechanistic explanation or hypothesis, an embodiment of the invention can nonetheless be operative and useful.

### BRIEF DESCRIPTION OF THE DRAWINGS

FIG. 1. Flowchart for montaging adjacent volumes. Step 1: The ILM and RPE for each volume are segmented. Step 2: Point clouds are created from the spatial coordinates of the segmented ILM and RPE. Step 3: Matching landmark points between Vm and Vs are identified. Step 4: The matching landmark points and point clouds are used to generate an initial rigid transformation between Vm and Vs. Step 5: The overlapping region between point clouds (green box in Step 4) is identified, and the transformation mapping the point clouds to each other in the overlapping region is computed to generate a final rigid transformation between Vm and Vs. Step 6: Strip-based registration generates a deformable registration between Vm and Vs. Step 7: The computed rigid and deformable registrations are applied to map overlapping volumes into the reference frame of Vs. The scale bars for Steps 1, 3, 5, and 7 are 1 mm. The distance between ticks is 0.2 mm for the z axis and 1 mm for the x and y axes in Steps 2, 4, 5, and 6. Each point cloud was down-sampled by a factor of 20 for visualization.

FIGS. 2A-2D. Montaged volume from HKU Eye Centre. FIG. 2A is an en-face OCT (Spectralis2, Heidelberg Engineering) projection of montaged volume. The FOV consists of nine overlapping single-volume FOVs (dotted squares). FIG. 2B is an en-face OCT without single-volume FOV overlayed. FIG. 2C is a B-scan corresponding to the blue line in FIG. 2B. FIG. 2D is a B-scan corresponding to the red line in FIG. 2B. Scale bars are 1 mm.

FIGS. 3A-3C. Montaged volume from Wills Eye Hospital. FIG. 3A is an en-face OCT (Cirrus HD-OCT, Zeiss) projection of montaged volume, consisting of three single-volume FOV (dotted squares). FIG. 3B is a B-scan image from position highlighted by the blue line in FIG. 3A. FIG. 3C is a B-scan image from position highlighted by the red line in FIG. 3A. Scale bars are 1 mm.

FIGS. 4A-4D. Montaged volume from Northwestern Memorial Hospital. FIGS. 4A and 4B are en-face images (Maestro2, Topcon), consisting of nine overlapping volumes (FOV noted by dotted squares). FIGS. 4C and 4D are B-scan images from the position highlighted by the blue and red lines in FIG. 4B. Bar: 1 mm.

FIGS. 5A-5C. Volumetric montage of 36 volumes from a single eye. FIG. 5A is an en-face image of the montaged volume, consisting of 36 overlapping single volumes, with each FOV highlighted by the dotted squares. FIG. 5B is an en-face OCT without outlines of individual volumes highlighted. FIG. 5C is a B-scan image from the position highlighted by the blue line in FIG. 5B. Scale bar: 1 mm.

FIGS. 6A-6E. Comparison of point cloud generation methods. FIG. 6A is when point clouds were generated with the ILM border, MSD was smaller after elastic registration. FIG. 6B is when point clouds were generated with the RPE border, MSD was smaller after elastic registration. FIG. 6C is when multi-layer point clouds were generated with the ILM and RPE border, with MSD smaller after elastic registration. FIG. 6D MSD for the RPE point cloud was comparable with the ILM point cloud. FIG. 6E comparable MSD for multi-layer point clouds when transformations were generated by single and multi-layer point clouds. Scale bars: 1 mm.

FIG. 7 is a schematic illustration of a system of the instant invention. CL is a collimator, FC fiber coupler, PC polarization controller, DC dispersion compensation, VA variable attenuator, TOF is a time-of-flight sensor, CMOS is a pupil camera, and M mirror

FIG. 8 is a structural block diagram illustrating components of computer system of the system.

### DETAILED DESCRIPTION

As used herein, “overlapping” refers to at least some portion of adjacent OCT volumetric images spatially overlap. Preferred overlapping optionally includes about 10%-30% of a volume that overlaps. This helps ensure there are sufficient feature matching points to obtain reliable mapping to a common reference frame. Aspects relevant to the mapping include: 1) maximizing correlation among landmark points, and 2) iterative closest point to align overlapping point clouds, and 3) accounting for accumulated drift in the volume montaging scheme through loop closure. We take advantage of the high volumetric resolution of OCT by generating point clouds representing the surface of the samples. With respect to overlapping, we acquire a plurality of volumes (in the exemplary embodiment, 8) with overlap between adjacent volumes (in the exemplary embodiment, about 20%) based on a trade-off between imaging acquisition time, data quality, and montaging accuracy. We detail example calculations based on the anterior segment of the mouse eye. Reports show that the diameter of the mouse eye varies between 3 mm (Park Hn, Qazi Y, Tan C, et al. Assessment of axial length measurements in mouse eyes. Optom Vis Sci. 2012; 89(3):296-303) and 4 mm (Treuting, P. M., Wong, R., Tu, D. C., & Phan, I. (2012). Special Senses: Eye. In Comparative Anatomy and Histology (pp. 395-418). Elsevier Inc). A diameter of 4 mm yields a circumference of 12.6 mm. For 8 volumes to cover the circumference, the average spacing between each pair of volumes is 1.57 mm. The lateral FOV of each of our volumes is 2.04 by 2.04 mm. This corresponds to an expected volume overlap of 23% between adjacent volumes. A diameter of 3 mm corresponds to an overlap of 43% between adjacent volumes. Thus, we expected at least a 23% overlap between consecutive volumes. We rounded 23% down to 20% to account for potential adjustments that needed to be made at each imaging position. The described methodology is not limited to the mouse eye, but is particularly useful for any of a variety of curved surfaces, and has been demonstrated on the human retina. It is reasonable to select a different overlap for alternative samples such that the relevant aspects mentioned above are accounted for.

The choice of the number of volumes is based on a trade-off between acquisition time, data size, and data quality. A few advantages of more acquisitions (and decreasing the FOV for each acquisition) are improved sampling resolution, edges of acquired volume closer to the focal plane, and the normal vector to tissue at the center of FOV oriented closer to the normal vector at the edge of the FOV. The disadvantages of more acquisitions are longer imaging times, increased data size, and additional data processing. We illustrate this example with acquisition of the full aqueous outflow pathway in mice. Thus, our goal is to acquire the minimal number of volumes necessary to image the full aqueous humor outflow pathway with high resolution and quality, finding that eight volumes were sufficient. We determine each volume's size based on the vis-OCT beam's depth of focus. Given our incident beam diameter of 2 mm, lens with a focal length of 25 mm, and a refractive index of 1.38, our depth of focus for a perfectly Gaussian beam is 307 μm. Our incident laser source is not perfectly Gaussian, so we have a greater focus depth. Assuming the eye is a sphere of diameter of 3.5 mm (approximately the size of the mouse eye in literature and our measurements), an OCT field of view of 2.04 mm by 2.04 mm, the height of surface of the eye at the center of the field of view differs by 328 μm the height at the edge of the field of view. Decreasing the number of volumes would have required a larger FOV per volume, which would have resulted in portions of the tissue being out of focus. Different implementations outside of the mice aqueous humor outflow pathway depend on the system properties of the OCT machine, but the logic in determining field of view and overlap remain the same.

We also ran a simulation testing the volume needed to generate stable geometric registrations between sets of 3 points randomly located in the cube. In this simulation, we randomly generated 3 points within a cube, with the cube length varying between 100 to 600 micrometers. These numbers correspond to roughly an overlap of 5 to 30%. We used 3 points since that is the minimal number of points needed to calculate a 3-dimensional geometrical transformation between a matched pair of points. We rotated each of these points by 45 degrees around the x-axis, y-axis, and z-axis of the simulated coordinate system, with the origin being one of the cube edges. For each of the rotated positions, we added Gaussian noise to the position of each of the points along each axis, with the standard deviation of the Gaussian noise set at 9 micrometers. We determined the standard deviation by manually selecting a landmark point 20 times and measuring the standard deviation of the selected locations. Next, we calculated the geometric transformation between the original position of the 3 points and the rotated points plus the Gaussian noise. The standard deviation of the rotation around the x-axis (with the mean being the set value of 45 degrees) is plotted (indicating 20% overlap is a good target). We repeated this simulation 4000 times.

The simulation results show that larger overlapping between adjacent volumes generates more stable transformations. The further away the points are, the less sensitive they are to noise. At 20% overlap, corresponding to a cube of roughly 400 microns, we see that the rate of stability improvement with increased cube dimension begins to reduce. In our montaging method, we use more than three points, so we would expect the stability of our transformation to be better than the simulation. Therefore, we chose 20% overlapping at least between adjacent volumes and eight volumes to cover the entire circumference. The 20% overlapping is readily fine-tuned and adjusted depending on the reliability to extract sufficient features between adjacent volumes.

In our data acquisition for mice anterior segment, this 20% overlapping was suitable for our data acquisition scheme. Of course, the methods provided herein accommodate and are compatible with a range of different overlaps, depending, for example, on other applications having different FOVs, resolutions, robot trajectories, etc.

An overlap of 20% is slightly less than the expected overlap area between acquired volumes, which ranges from 23 to 43%, assuming an eye diameter ranging from 3 mm to 4 mm and a lateral field of view of 2.04 mm. We note that the percentage of overlap between adjacent volumes depends on the acquisition scheme, but the overlapping volume must provide adequate shared landmarks between the volumes.

Until now, volumetric montaging works have accounted for translation between volumes by registering en-face projections and rotation by rotating individual B-scans in each volume to maximize cross-correlation between B-scans. However, en-face projections exhibit distortions when the curvature of the tissue relative to the incident beam varies. Additionally, rotating individual B-scans distorts structures nonlinearly. We addressed these issues by taking the structures as a whole and volumetrically registering 3D point clouds representing each volume. This is particularly relevant for OCT imaging of a curved tissue, such as the retina.

Nearly all existing OCT montaging algorithms register en-face projection images [62, 63]. However, en-face projections become nonlinearly distorted relative to each other when their curvature relative to the incident beam is significantly different from each other, as in our situation. To circumvent this obstacle, we used 3D volumetric surface point cloud data to volumetrically montage all datasets, which does not suffer the same angular distortion.

“Surface topology parameter” refers to a parameter describing the morphology of the retina useful in a characterization, including a diagnostics, of the retina. It may include, for example, a parameter that can characterize curvature, such as a best-fit 3D surface, 2D linear equations over selected linear regions of the eye, surface roughness, and the like.

“Increasing” or “expanding” generally refers to the capability of the instant methods and systems to provide an increased OCT imaging area that does not unduly suffer from degradation or distortion. This is accomplished by montaging of individual OCT volumetric images. In a quantitative context, “increasing a FOV” will depend on the number of the plurality of OCT volumetric images and the amount of overlap. As described herein, however, the increase can be substantial, including to cover the entire tissue of interest, including the entire retina, the entire eye, and especially including the outer-most circumferential regions of a curved surface. The upper bound of range increase is unlimited, only bounded by time constraints of user. For the retina, the field of view may be expanded up to 200 degrees. Smaller increases in range are possible pending data acquisition parameters.

Imaging complex, non-planar anatomies with optical coherence tomography (OCT) is often limited by the optical FOV achieved in a single volumetric acquisition. Specifically, limitations in mechanical scan angle and illumination orientation constrain the imaging volume for most OCT systems. The present disclosure provides systems and methods for the acquisition of a plurality of OCT volumetric images and the construction of a compound volumetric image by montaging of multiple individual OCT volumetric images via point cloud imaging of a surface of each individual volumetric image. In particular, aspects of the present disclosure include (i) processes for mapping, based on the geometry of an object to be imaged (e.g. an object having a complex, non-planar anatomy—such as the full anterior segment of the eye), a plurality of image capture locations for an OCT sample arm during imaging of the object to be imaged, and (ii) processes for constructing a compound volumetric image from a plurality of individual OCT volumetric images, not constrained by any system design, acquired from a plurality of different image capture locations.

Determining common matching landmark points between overlapping images with different fields of view. Whereby, features in the enface projection are matched between different volumetric images. In particular, a set of features is selected in one image. For each point, a local region is taken, and the cross-correlation between that region and the second image is calculated. Matching points are determined by maximizing cross-correlation. Based on the relative coordinates of matching points and cross-correlation values, a set of points is selected as the matching set of points. Thus, a strategy for identifying common landmark points between different volumetric images is provided, with the constraint that the features have similarities between volumetric images.

Strip-based deformable registration following volumetric rigid registration, where relative eye motion between adjacent volumes is calculated. In the moving volumetric image, local sub-volumes corresponding to a strip of the larger volume are selected. Each strip is volumetrically registered to the second volume. Next, discrepancies between the registration generated by the sub volume and global rigid registration are used for a deformation map corresponding to deformable registration. Thus, a method to correct for local deformations not accounted for by translation and rotation is provided.

The methods provided herein may be implemented in a computer-readable medium, including software that provides instructions to a processor for carrying out the methodology. In particular, the software provides a volumetric montage of OCT datasets in 3D given volumetric images and description of which volumes are expected to overlap. As noted in Example 1, the software may use point cloud registration techniques used by computer vision field. The software may be implemented in commercially available platforms, such as MATLAB or python.

Relevant applications for the methods and systems provided herein include, but are not limited to: Peripheral retina OCT (micron scale resolution) clinical imaging; Early diagnosis of retinal diseases impacting the eye periphery; Point cloud-based eye tracking (gaze tracking); Expanded FOV OCT imaging: Mapping out the curvature of myopic eyes; Multiple sub-volumes can be used as motion correction for other volumes; Locating posterior staphylomas in the eye periphery.

Advantages of the methods and systems provided herein include: Combination of smaller FOVs allows avoidance of mirror artifacts present in other large FOVs achieved with optics; Large FOV with high-density sampling achieved, enabling maximization of OCT resolution; Point cloud-based registration much faster than using raw images; Applicable on existing hardware available in clinic; no additional expensive device needed; Accounts for eye rotation; existing methods usually only account for translation. There can be particular value with the instant technology with respect to the ability to obtain early diagnosis and management of disease conditions that otherwise are not reliably caught by conventional OCT methods and systems.

In the following description, numerous specific details of the devices, device components, and methods of the present disclosure are set forth in order to provide a thorough explanation of the precise nature of the invention. It will be apparent, however, to those of skill in the art that the invention can be practiced without these specific details. The invention can be further understood by the following non-limiting examples.

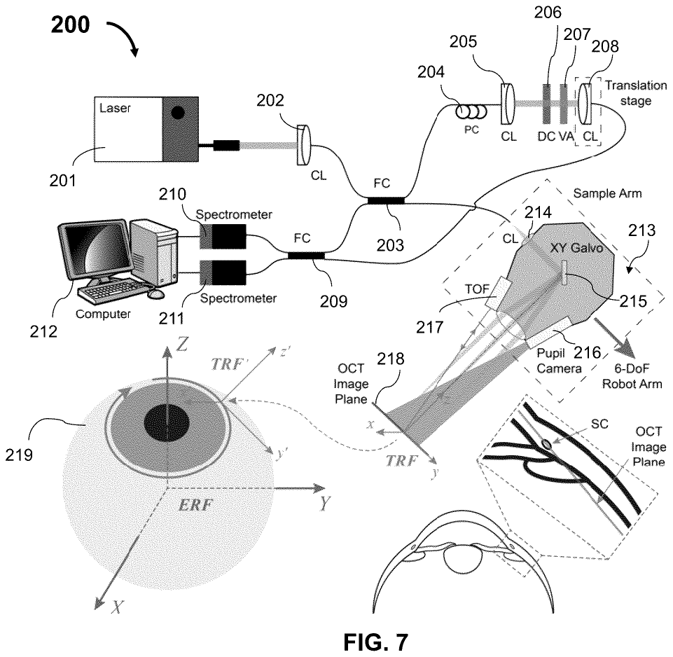

An example of a system for constructing an expanded FOV of an object from a plurality of OCT volumetric images is illustrated in FIG. 7 (adapted from WO 2024/226530, which is specifically incorporated herein for the systems, including OCT imaging systems and processing circuitry). Briefly, FIG. 7 illustrates a robotic visible light-optical coherence tomography (vis-OCT) imaging system. The OCT imaging system 200 includes a light source 201, that provides an output that is transmitted to a 90:10 fiber coupler (FC) 203 via collimator 202. FIG. 7 illustrates the system is optionally robotic controllable, with 10% sent to the sample arm 213 and 90% to the reference arm. To filter the output of the light source and minimize light outside the visible spectrum, the output can be passed through a dichroic mirror, a bandpass filter, and a spectral shaping filter. The reference arm includes a polarization controller 204, collimator 205, a dispersion compensator 206, a variable attenuator 207, and an additional collimator 208 in a translation stage. Input to the sample arm 213 from the fiber coupler 203 is collimated by collimator 214 before being scanned by a galvanometer 215 and focused onto the sample at the OCT image plane 218. Back-scattered light from the sample is passed through the fiber coupler 203 and interfered with the light from the reference arm in a 50:50 fiber coupler (FC) 209. Two well-calibrated spectrometers 210 and 211 detected the interferogram simultaneously for balanced detection. A compact sample arm 213 is mounted on a high-precision six-axis robotic arm. A pupil camera 216 and a time-of-flight (TOF) proximity sensor 217 are integrated with the sample arm 213. The pupil camera 216 is configured to provide an initial image for locating eye 219, and the TOF sensor 217 is utilized to prevent the sample arm 213 from colliding with the sample, i.e. eye 219.

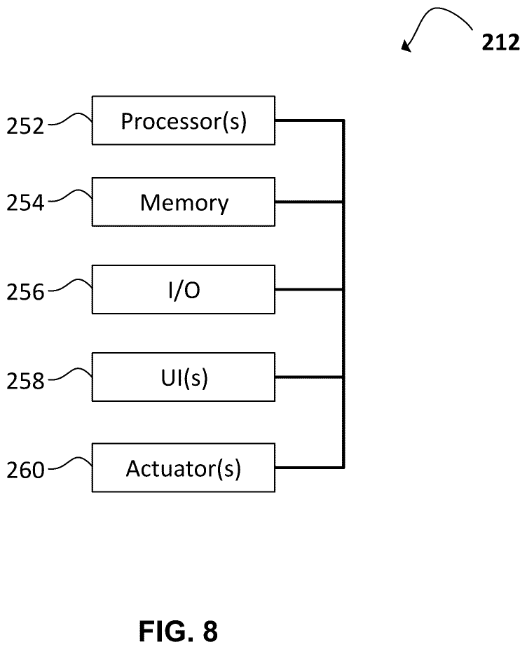

The system 200 additionally includes computer system 212. FIG. 8 is a block diagram illustrating various components of the computer system 212. The computer system 212 includes one or more processors 252, memory 254, one or more input/output devices 256, one or more user interfaces 258, and one or more actuators 260.

The one or more processors 252 can include one or more distinct processors, each having one or more cores. Each of the distinct processors can have the same or different structure. The one or more processors 252 can include one or more central processing units (CPUs), one or more graphics processing units (GPUs), circuitry (e.g., application specific integrated circuits (ASICs)), digital signal processors (DSPs), and the like. The one or more processors 252 can be mounted to a common substrate or to multiple different substrates.

The one or more processors 252 are configured to perform certain functions, methods, or operations (e.g., are configured to provide for performance of functions, methods, or operations). The one or more processors 252 can perform operations embodying a function, method, or operation by, for example, executing code (e.g., interpreting scripts) stored on memory 254 and/or trafficking data through one or more ASICs. The one or more processors 252, and thus the computer system 212, can be configured to perform, automatically, functions, methods, and operations disclosed herein. Therefore, the computer system 212 can be configured to implement methods described herein. For example, when the present disclosure states that a method or device performs task “X” (or that task “X” is performed), such a statement can be understood to disclose that computer 212 is configured to perform task “X”. The computer system 212 is configured to perform a function, method, or operation at least when the one or more processors 252 are configured to do the same.

Memory 254 can include volatile memory, non-volatile memory, and any other medium capable of storing data. Each of the volatile memory, non-volatile memory, and any other type of memory can include multiple different memory devices, located at multiple distinct locations and each having a different structure. Memory 254 can include remotely hosted (e.g., cloud) storage. Examples of memory 254 include a non-transitory computer-readable media such as RAM, ROM, flash memory, EEPROM, any kind of optical storage disk such as a DVD, a Blu-Ray® disc, magnetic storage, holographic storage, a HDD, a SSD, any medium that can be used to store program code in the form of instructions or data structures, and the like. Methods, functions, and operations described herein can be fully embodied in the form of tangible and/or non-transitory machine-readable code (e.g., interpretable scripts) saved in memory 254.

The one or more input-output devices 256 can include any component for trafficking data such as ports, antennas (i.e., transceivers), printed conductive paths, and the like. Input-output devices 256 can enable wired communication via USB®, DisplayPort®, HDMI®, Ethernet, and the like. Input-output devices 256 can enable electronic, optical, magnetic, and holographic, communication with suitable memory 254. Input-output devices 256 can enable wireless communication via WiFi®, Bluetooth®, cellular (e.g., LTE®, CDMA®, GSM®, WiMax®, NFC®), GPS, and the like. Input-output devices 256 can include wired and/or wireless communication pathways.

The one or more user interfaces 258 can include displays, physical buttons, speakers, microphones, keyboards, and the like. The one or more actuators 260 can enable the one or more processors 252 to control other components of the system 200. For example, the one or more actuators, which may be remotely located from the other components of the computer system 212, can be configured to control the light source 201 and a robotic arm 220.

The computer system 212, and its various components, can be distributed. For example, some components of the computer system 212 can reside in a remote hosted network service (e.g., a cloud computing environment) while other components of the computer system 212 can reside in a local computing system. The computer system 212 can have a modular design where certain modules include a plurality of the features/functions shown in FIG. 8. For example, I/O modules can include volatile memory and one or more processors. As another example, individual processor modules can include read-only-memory and/or local caches.

### Example 1: Volumetric 3D Montaging of Optical Coherence Tomography Volumes

Optical coherence tomography (OCT) has transformed clinical eye care by providing high-resolution volumetric imaging of the retina. Recently, ultrawide-field-of-view (FOV) OCT played an increasingly significant clinical role; however, most clinical OCT systems offer only a rather limited FOV. We increase the FOV of clinical OCT by volumetrically montaging multiple OCT datasets in three dimensions (3D). Volumetric montaging is performed by representing the internal limiting membrane (ILM) and retinal pigment epithelium (RPE) in each volume as point clouds and using these point clouds to compute transformations that map each volume to a common reference frame. The methodology is validated using datasets from three institutions with different OCT hardware and data-acquisition procedures. Using the mean surface distance between point clouds, we find the error in montaging is less than the lateral pixel size. The methods provided herein facilitate existing clinical OCT to achieve ultrawide FOV imaging without any hardware modification.

Optical coherence tomography (OCT) is a non-invasive imaging modality that has revolutionized the management of nearly all ocular diseases [1-3]. OCT detects the interferogram from short-coherence-length light to achieve depth-resolved information with microscopic axial resolution. Transversely scanning the beam in the lateral dimension further generates three-dimensional (3D) volumetric images. The FOV is determined by the lateral scanning size, and the imaging depth is determined by the spectrometer design or the swept-source laser used [4, 5]. Although the FOV can be expanded by mechanically scanning a larger area, this comes at the expense of sparser sampling [6-8] and fringe washout due to fast mechanical scanning [9]. In highly curved eyes with long axial eye length, OCT imaging depth is often unable to capture the full FOV without mirror artifacts [10], [11].

Because many retinal diseases manifest in the peripheral retina, it is clinically important to expand OCT's FOV. Pathological myopia is a condition in which an increased FOV is beneficial, helpful in locating posterior staphylomas, characterizing abnormal posterior scleral curvature, dome-shaped macula, identifying retinoschisis, retinal breaks, as well as potentially detecting retinal detachment [12-15]. Additionally, expanding the FOV to include the peripheral retina improves the management of diabetic retinopathy and vascular diseases. Recent studies suggest that peripheral retinal nonperfusion helps grade diabetic retinopathy [16, 17]. Consequently, much research has focused on increasing OCT's FOV.

One method to increase the FOV is to redesign OCT's optical and hardware components. Researchers have reported using custom-designed widefield lenses to increase the FOV to over 90 degrees [18, 19]. Additionally, robotic OCT has been used to increase the FOV and to locate peripheral regions of interest [20-24]. Unfortunately, many of those customized OCTs are not yet commercially available or are prohibitively expensive. The vast majority of clinical OCTs still have limited FOVs.

Another method to increase the FOV is to montage multiple overlapping volumetric datasets in existing OCTs. The primary advantage of montage-based designs over hardware-based designs for increasing the FOV is the higher scan density. To image a wider FOV in a short time, which is necessary for patient comfort, the lateral sampling density of widefield OCT becomes increasingly sparse. Less-dense sampling results in reduced signal quality and underutilization of OCT's high resolution. Without sufficiently dense scanning, OCT may fail to visualize smaller retinal features adequately [6-8].

The primary drawback of montage-based methods is the artifacts from the distortion caused by projecting spherical retinal anatomy onto planar projections from multiple OCT volumes [25, 26]. Despite this drawback, montaging remains an attractive way to increase the FOV of existing clinical OCTs.

Several investigators have applied montaging to viewing retinal projections across a wider region of the eye. Most studies on increasing the FOV focused on stitching two-dimensional (2D) en-face projection images of the retina [27, 28]. Other investigators have attempted to montage 3D volumes; however, these studies were often performed ex vivo and only accounted for coordinate translations between volumes [29-31]. Due to the nature of eye motions between OCT scans, rotation between volumes must be accounted for. Mori et al. used montaging to visualize retinal cross-sections over a wider FOV. However, they only merged individual B-scans rather than the full volume [32]. Ji et al. developed a method for 3D montaging volumetric images of the skin by flattening each volume and determining the translation between the flattened volumes [33]. However, flattening the volumes distorts the 3D topology of the structure, which is particularly important for understanding the retina's curvature. So far, there is a lack of a method for 3D volumetric montaging of clinical retinal OCTs.

In this study, we volumetrically montage up to 36 OCT volumes in 3D to increase the FOV of clinical OCTs. For 3D volumetric registration, we represent each volume as a point cloud. We determine the rigid transformations and displacement vector fields needed to map all volumes to a common reference frame. Compared with previous attempts to combine OCT volumes, this study factors in rotations between acquired volumes. We demonstrate the effectiveness of our method using datasets acquired from multiple institutions, with varying data-acquisition patterns and OCT devices.

Methods: Data Acquisition: We imaged three eyes with high myopia and one normal eye from four subjects. Nine 3D OCT volumes were acquired from each eye with a spectral-domain OCT (Spectralis2TM instruments, Heidelberg Engineering, Germany). Each volume is acquired with a 30°×28° FOV, which is approximately 10 mm×10 mm in the eye. Each volume consists of 135 B-scans, each with 496 A-lines. After acquiring the fovea-centered scan, the center of focus is translated by ±10° in the horizontal direction to acquire two more volumes. From these three positions, the center of focus is further translated by ±10° in the vertical direction for 6 additional volumes.

We imaged eight eyes from seven subjects. Seven healthy subjects with refractive errors ranging from 0 to-6 diopters underwent spectral-domain OCT imaging (Cirrus HD-OCT, Zeiss) using the macular cube and optic disc cube scanning protocols. Each volume consisted of a 6.0 mm×6.0 mm FOV (approximately 20°×20°), with 200 B-scans and 200 A-lines per B-scan. We acquired up to 36 volumes with approximately 20% adjacent overlap per volume, covering FOVs of up to 100 degrees per eye. For the 36-volume acquisition, 18 macular cube and 18 optic disc cube volumes were acquired.

We imaged 26 eyes from 13 subjects. We imaged both eyes of each subject using spectral-domain OCTA (Maestro2, Topcon) with a 6.0 mm×6.0 mm FOV, 360 B-scans, and 360 A-lines per B-scan. We first obtained a macula-centered scan using internal fixation targets. We instructed subjects to look slightly off-center at the internal fixation target until only 30% of the first macula scan was included. We acquire volumes superior, inferior, temporal, superotemporal, and inferotemporal to the macula. This procedure was repeated with an optic nerve-centered scan with additional volumes in the nasal, superonasal, and inferonasal directions, for 9 scans.

Volumetric Montaging Overview: After acquiring multiple OCT volumes with partially overlapping regions, we merge the 3D structural data volumetrically. The challenge of montaging multiple datasets reduces to determining the relationship between the local reference coordinates of each volume and a common global coordinate, which is the coordinate system of the first OCT volume (usually the centermost volume).

For the local reference coordinate of each OCT volume Vn, we define the x-axis as the fast-scanning axis, the y-axis as the slow-scanning axis, and the z-axis as the axial axis. Given two adjacent 3D dataset volumes, Vs and Vm, we first determine their relative positions and orientations. After that, we transform the coordinate of Vm (moving volume) to the coordinate of Vs (stationary volume).

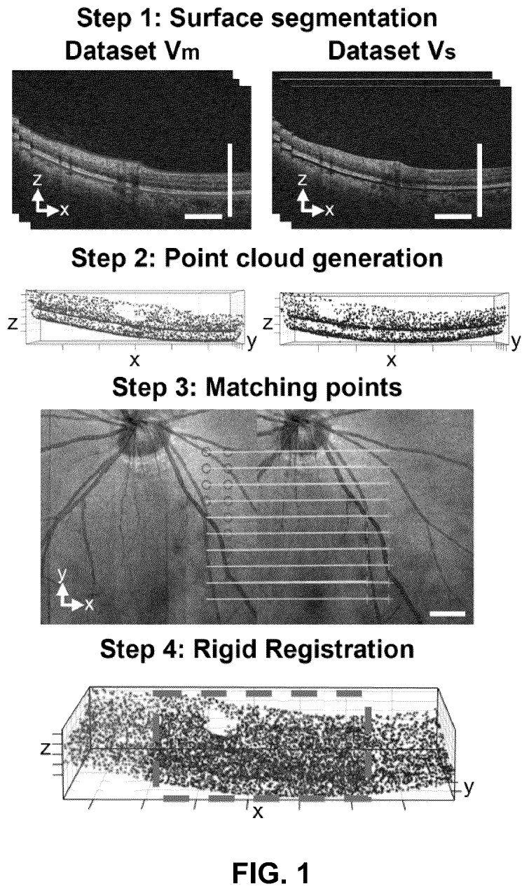

FIG. 1 shows a flowchart for montaging partially overlapping volumes Vs and Vm. In Step 1 (“Surface segmentation”), we segment the internal limiting membrane (ILM) and retinal pigment epithelium (RPE) for each volume. In Step 2 (“Point cloud generation”), we represent the 3D coordinates of the ILM and RPE segmentation as a point cloud. In Step 3 (“Matching points”), we identify common landmark points in the en-face projection of the two volumes. In Step 4 (“Rigid Registration”), we use the 3D coordinates of the landmark points to estimate an initial rigid transformation that aligns Vm to Vs. In Step 5 (“Overlap Registration”), we identify the overlapping region between Vs and Vm sharing the same x and y coordinates in the Vs coordinate system. We register the point clouds of Vs and Vm in the overlapping region to refine the rigid registration generated in Step 4. In Step 6 (“Elastic Registration”), we divide the Vm point cloud into strips. We optimize the transformation of each strip to the Vs point cloud to generate a displacement vector field used for elastic registration of Vm to Vs. In Step 7 (“Merge Volumes”), we apply the rigid transformation and displacement vector field to the OCT volume corresponding to Vm to generate a composite merged OCT volume with Vs. More details for each step are described in the following sections.

Step 1: Surface Segmentation: We use a U-Net-based machine learning model to segment the ILM and RPE [34]. To create the model, we fine-tuned the existing OCT-Retinal Layer Segmenter model using 20 manually segmented B-scans. We train the model on an NVIDIA GTX 3090 Ti GPU with 24 GB of VRAM. During training, we minimize the categorical cross-entropy function

$$L = - {\sum }_{i = 1}^{2} {l}_{i} log ( {p}_{i} ) , \qquad ( 1 )$$

where li is the true label for sub-layer i and pᵢ is the predicted probability of sub-layer i from the model output. In our case, the two sub-layers comprised the retina between the ILM and RPE, as well as the background. We define the ILM and RPE as the inner and outer layers of the segmented retina. Step 1 in FIG. 1 shows the segmented ILM for two B-scans.

Step 2: Point Cloud Generation: We generate a point cloud of the 3D coordinates of the segmented ILM and RPE surfaces. We define the point cloud as the spatial coordinates of the ILM and RPE boundaries across all B-scans. Alternatively, only one boundary, either ILM or the RPE, could be used when segmentation is challenging.

For Vm, we define n as the number of A-lines per B-scan, and b as the number of B-scans per OCT volume. We define xᵢ as the x coordinate for each A-line within a B-scan and yⱼ as the y coordinate for each B-scan. We let F(xᵢ, yⱼ) and G(xᵢ, yⱼ) be the axial depths of the ILM and RPE at the lateral coordinate (xᵢ, yⱼ). Using these definitions, the point cloud M for volume V_{M} is

M={(xᵢ,yⱼ,F(xᵢ,yⱼ)),(xᵢ,yⱼ),G(xᵢ,yⱼ)))|i=1, . . . ,n and j=1, . . . ,b}, (2)

We define the point cloud S for Vs identically.

Step 3: Matching Points: We determine the spatial coordinates of matching landmark points in the en-face projections of Vs and Vm. Step 3 of FIG. 1 shows matching landmark points in the en-face projections of Vm and Vs. We generated a set of 2D grid points in the en-face image of Vm, with the grid located either in the top, left, right, or bottom region of the en-face projection of Vm, with the choice of region corresponding to where the en-face projections of Vm and Vs overlapped. For the example in Step 3 of FIG. 1, the points located at the open circles correspond to the set of 2D grid points mentioned above. In this case, the grid points are located on the right because the right side of the en-face projection of Vm overlapped with the en-face projection of Vs.

The points toward the right side of Step 3 of FIG. 1 correspond to the matching points in the en-face projection of Vs. We use normalized image screen coordinates to describe the locations of the grid points, where the top-left point of the en-face projection corresponds to (0,0), the bottom-right to (1,1), the top-right to (1,0), and the bottom-left to (0,1). When the 2D grid points were in the left or right region, the grids consisted of two columns of ten points with a separation of (0.1, 0) between points in the x direction and (0, 0.075) between points in the y direction. The top left point of the left grid was located at (0.225, 0.2), and the top right point of the right grid was located at (0.775, 0.2). The descriptions of the grid points in the top and bottom regions are analogous, except that the x- and y-axes are swapped.

For each grid point, we took a 0.25 normalized width (25 percent of the en-face projection width) by 0.25 normalized height region from the en-face projection of Vm centered on the point. We computed the 2D normalized cross-correlation between this region [37] and the en-face projection of Vs. We assigned the coordinate in the en-face projection of Vs with the maximum normalized cross-correlation as the matching point to the grid point in the en-face projection of Vm.

Step 4: Rigid Registration: We obtain an initial estimate of the rigid transformation that maps the reference frame of Vm to that of Vs by computing the transformation that maps the spatial coordinates of common landmark points between Vm and Vs. Rigid transformations preserve the spatial relationship between points, including translations, rotations, and reflections.

Given a total number of p matching landmark points, we define the set of matching landmark points for Vm as

LM={(xᵢ,yᵢ)|i=1, . . . ,p}.

Where (xᵢ, yᵢ) is the lateral position of the i^{th} landmark point.

We define the set of 3D coordinates corresponding to these landmark points for Vm as

L_{M}={(xᵢ,yᵢ,F(xᵢ,yᵢ)),(xᵢ,yᵢ,G(xᵢ,yᵢ))|(xᵢ,yᵢ)∈LM}, (4)

Similarly, we specify the matching feature points for Vs as LS, and the 3D coordinates of these points as L_{S}.

Next, we use the M-estimator sample consensus (MSAC) algorithm [38] to compute the transformation Tᵢₙᵢₜ mapping L_{M} to L_{S}, as shown in Step 4 of FIG. 1.
Step 5: Overlap Registration: We refine the transformation Tᵢₙᵢₜ by applying the iterative closest point (ICP) algorithm to the subset of M and S within the spatially overlapping region between M and S (dashed box in Step 4 of FIG. 1). To determine the spatially overlapping region, we find the polygon bounding S by computing the alpha shape associated with S[40]. We let OM be the set of points of Tᵢₙᵢₜ (M) with x and y coordinates enclosed within the polygon bounding S. Subsequently, we define the points in M within the overlapping region as

M′={(xᵢ,yᵢ,F(xᵢ,yᵢ)),(xᵢ,yᵢ,G(xᵢ,yᵢ))|Tᵢₙᵢₜ(xᵢ,yᵢ,F(xᵢ,yᵢ))∈OM and Tᵢₙᵢₜ(xᵢ,yᵢ,G(xᵢ,yᵢ))∈OM}. (5)

We determine S′, the region of S within the overlapping region, in an identical manner to M′. Finally, we apply the iterative closest point (ICP) algorithm to estimate the transformation T that maps M′ to S′. Mathematically, the ICP algorithm minimizes an error function E

$$E ( T , {S}^{′} , {M}^{′} ) = {\sum }_{i = 1}^{N} min ( {‖ T ( {M}_{i}^{′} ) - {S}^{′} ‖}^{2} ) , \qquad ( 6 )$$

where the index M′i is the i^{th} point of M′ and ∥(T(M′ᵢ)−S′∥²) is the Euclidean norm of (T(M′ᵢ)−S′). Here, we let min stand for minimum, meaning that the closest point in S′ to T(M′ᵢ) was used to calculate Euclidean distance. Step 5 of FIG. 1 shows T(M′) in blue and S′ in red, with the two point clouds aligning closely in space.
Step 6: Elastic Registration: Due to eye motion, rigid transformations do not perfectly align the point clouds M and S. We adopt strip-based registration methods commonly used in adaptive optics OCT to compute a deformation vector field that best aligns the two volumes [41, 42]. Letting a and b be the upper and lower bounds for the y coordinates of the strip, we define the strip of M with y coordinates within these bounds as

M_{ab}={(xᵢ,yᵢ,zᵢ)|(xᵢ,yᵢ,zᵢ)∈M and a≤yᵢ≤b}. (7)

Next, we use the ICP algorithm to find the transformation T_{ab} mapping M_{ab} onto its associated region in S (Step 6 in FIG. 1). To prevent overfitting, we only used the subset S_{ab} of S consisting of the closest points in S to T(M_{ab}). As with the calculation of the rigid transformation, the ICP algorithm minimized the error function E defined as

$$E ( {T}_{ab} , {S}_{ab} , {M}_{ab} ) = {\sum }_{i = 1}^{N} min ( {‖ ( {T}_{ab} ( {M}_{ab , i} ) - {S}_{ab} ‖}^{2} ) , \\ where {M}_{ab , i} is the {i}^{th} element of {M}_{ab} . \qquad ( 8 )$$

Following the registration of each sub-strip of M onto S, we define the transformation Tₛ, consisting of a piecewise set of transformations aligning strips of M onto S. Letting a and b be the lower bounds for the first strip and c and d be the upper bounds for the last strip, we define Tₛ as

$${T}_{s} ( x , y , z ) = \{ \begin{matrix}{T}_{ab} ( x , y , z ) , a \leq y < b \\ … , … \\ {T}_{cd} ( x , y , z ) , c \leq y < d\end{matrix} . \qquad ( 9 )$$

The displacement vector field D for each spatial coordinate (x, y, z) in the original frame is defined as

D(x,y,z)=Tₛ(x,y,z)−T(x,y,z), (10)

Conceptually, D is the displacement of the coordinates of T(M) necessary to align T(M) with the coordinates of S.
Step 7: Merge Volumes: Following the determination of T and D, we map the volumetric dataset of Vₛ and Vₘ to the common reference frame associated with Vₛ to form a merged volume V. As the merged volume I has the same reference frame as Vₛ, we applied no transformations or deformations to Vₛ. For volume Vₘ, we updated its contribution to the intensity in V as

V(x′,y′,z′)=(Vₘ(x,y,z)+Vₛ(x′,y′,z′))/2 if T(x,y,z)+D(x,y,z)=(x′,y′z′). (11)

In other words, if coordinate (x, y, z) in Vₘ mapped to (x′, y′, z′) in Vₛ after applying the rigid and deformable transformation to Vₘ, then the contribution of Vₘ to the merged volume V at the coordinate (x′, y′, z′) is Vₘ (x,y,z).

In instances where more than two volumes contributed to the intensity V(x,y,z), we take the mean contribution from all the volumes.
Step 7 in FIG. 1 shows the process of merging the volumetric data. The upper left panel of step 7 shows a B-scan of Vₘ in its local reference frame. The rigid transformation T and displacement D are applied to map the Vₘ into the reference frame of Vₛ. The lower left panel of Step 7 shows a B-scan of Vₛ in its local reference frame. The merged dataset of Vₘ and Vₛ combines the structural data of Vₘ and Vₛ in the reference frame of Vₛ, with averaging of data in the overlapping region (bottom right panel of Step 7). In cases with three or more datasets, each volume is weighted equally in overlapping regions.

To generate en-face projections of the merged volume, we compute the mean of the 30 voxels with the highest intensity in the depth dimension at each lateral position. We choose not to use all voxels in the depth dimension for the en-face projection because each lateral position has a different number of voxels corresponding to background noise, depending on how many single volumes mapped to that lateral location.

Assessing Montaging Error: Following 3D volumetric montaging, we generate a cumulative point cloud comprising the union of every volume's individual point cloud mapped into the common global coordinate. Suppose there exist point clouds A through N, with T_{A} being the rigid transformation for A into the global coordinate and DA being the deformation vector field, then we define the cumulative point cloud CP as

CP={(T_{A}(A)+D_{A}(A)), . . . ,(T_{N}(N)+D_{N}(N))}. (12)

Next, we remove the point cloud corresponding to volume V_{A} from the cumulative point cloud. We determine the overlapping region between the point cloud (T_{A}(A)+D_{A}(A)) and the cumulative point cloud of the other volumes CP−(T_{A}(A)+D_{A}(A)) using the methodology described in Step 5. For every point of (T_{A}(A)+D_{A}(A)) in the overlapping region, we measure the distance between the point and the closest point in CP−(T_{A}(A)+D_{A}(A)). We define the mean surface distance (MSD) as the mean distance between points in (T_{A}(A)+D_{A}(A)) and the nearest point in CP−(T_{A}(A)+D_{A}(A)). Additionally, we define the median surface distance as the median distance between points in (T_{A}(A)+D_{A}(A)) and the nearest point in CP−(T_{A}(A)+D_{A}(A)). Similarly, we define the 95% Hausdorff distance as the 95th percentile of the distance between points in (T_{A}(A)+D_{A}(A)) and the nearest point in CP−(T_{A}(A)+D_{A}(A)). We repeat this process for every volume.

Statistical Analysis: All statistical comparisons between two groups are done with a paired T-test, and all comparisons between more than two groups were done with a one-way ANOVA. We reported all values as mean±standard deviation.

RESULTS: Montaging datasets from multiple institutions

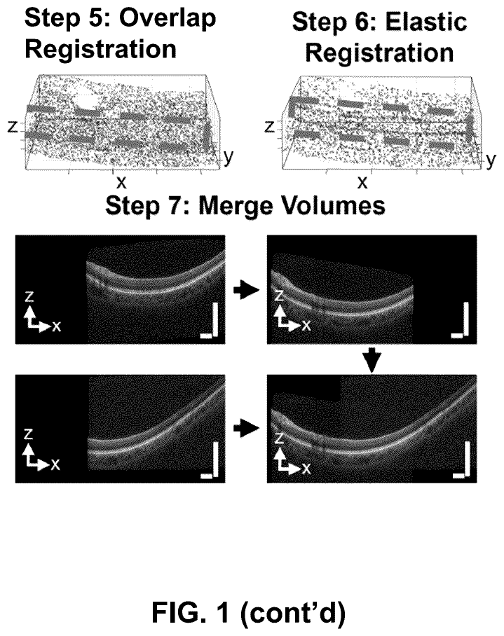

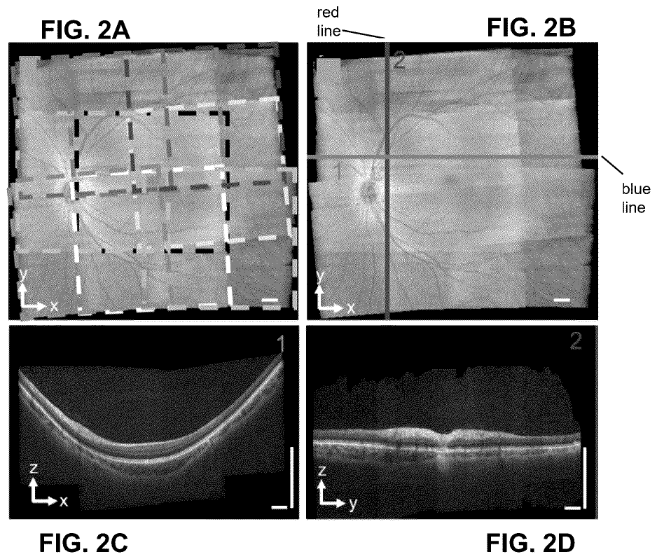

We volumetrically montage four subjects imaged at the HKU Eye Centre. FIGS. 2A and 2B show the en-face projection for the montaged dataset from one subject, with the nine dotted squares highlighting the FOV for each original volume shown in FIG. 2A. The FOV of the montaged volume in FIG. 2B is 18.2 mm×17.4 mm and 3.2 mm deep. The FOV for a single volume in FIG. 2A Is 9.3 mm×9.3 mm and 1.9 mm deep.

FIG. 2C shows a B-scan image from the montaged volume from the position highlighted by the blue line in FIG. 2B. FIG. 2D shows the B-scan image from the position highlighted by the red line in FIG. 2B. Both B-scan images show continuous retinal layers in the final montage volume. We compute the average MSD between volumes as 38.3±14.0 μm (n=4 eyes) after rigid registration and 26.8±13.3 μm after elastic registration.

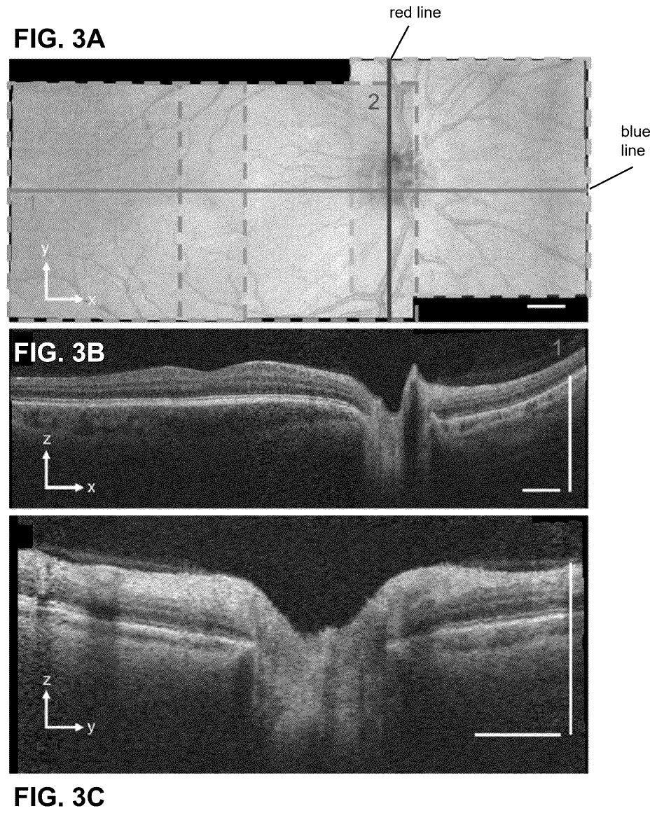

We test our montaging algorithms in eight eyes imaged at Wills Eye Hospital. FIG. 3A shows the montaged en-face projection, with dotted squares highlighting the FOV for each volume. The FOV for the montaged volume is 15.0 mm×6.8 mm and 2.6 mm deep. The FOV for each volume is 6.0 mm×6.0 mm and 2.0 mm deep.

FIG. 3B shows the B-scan image from the montaged volume from the position highlighted by the blue line in FIG. 3A. This B-scan integrates data from three volumes. FIG. 3C shows B-scan image from the position highlighted by the red line in FIG. 3A. Both B-scans contain data from multiple original volumes, none of them exhibit discontinuities at the boundary between volumes.

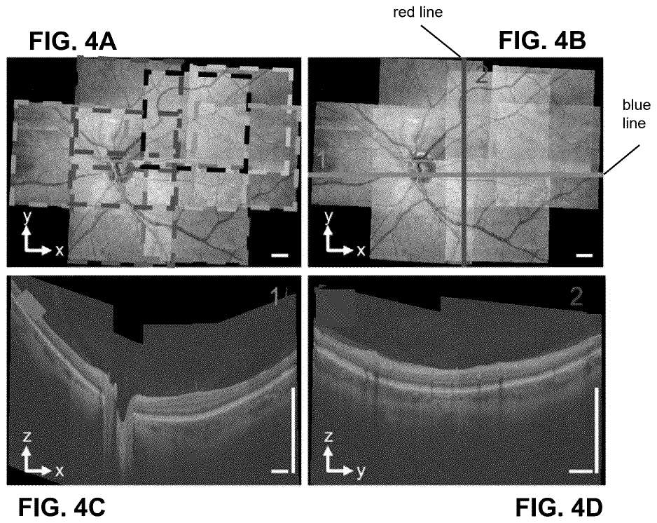

We montage 18 eyes at Northwestern Memorial Hospital, with eight of the 26 eyes excluded due to significant motion artifacts. FIGS. 4A and 4B show the montaged en-face projections, with dotted squares depicting the FOV for each volume in FIG. 4A. The FOV for the montaged volume was 17.2 mm×12.2 mm and 3.5 mm deep. The FOV for each volume was 6.0 mm×6.0 mm and 2.3 mm deep.

FIG. 4C shows the B-scan image of the montaged volume corresponding to the blue line in FIG. 4B, covering data from six volumes. FIG. 4D shows the B-scan image from the position highlighted by the red line in FIG. 4B, incorporating data from six volumes. Both B-scan images exhibit no discontinuities at the boundary between volumes.

B. Large Field of View Montaged with 36 Volumes

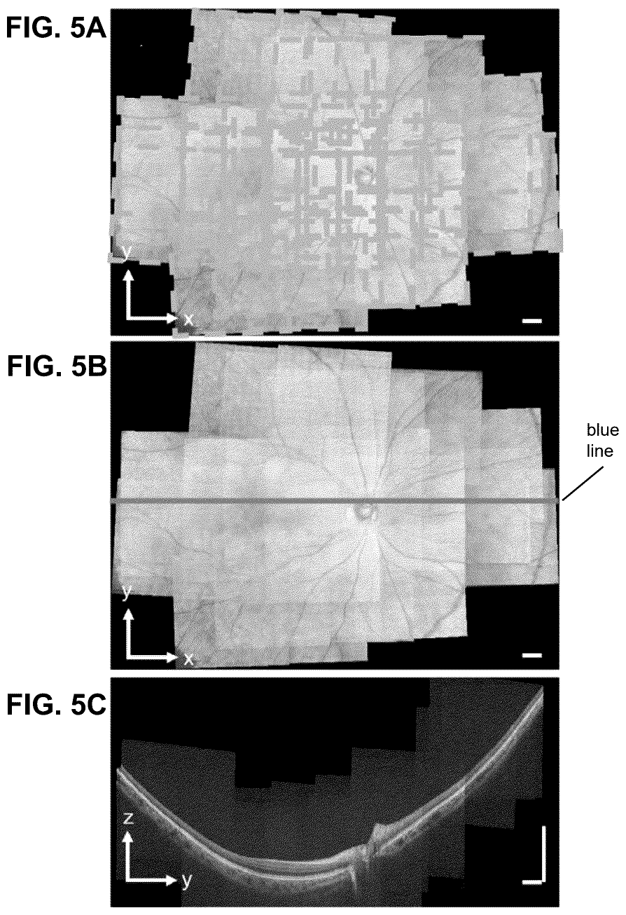

We acquired 36 volumes from a single eye at Wills Eye Hospital. FIG. 5A shows the en-face image of the montaged volume, with the dotted green squares highlighting FOVs of individual volumes. FIG. 5B shows the en-face image without the dotted boxes. The montaged volume covers 23.6 mm×17.2 mm FOV and 4.8 mm deep.

FIG. 5C shows the B-scan image of the montaged volume from the position highlighted by the blue line in FIG. 5B, incorporating data from 23 overlapping single volumes.

After rigid registration, we find that the MSD between volumes in this eye was 20.2+18.3 μm (n=36 individual volumes), the 95% Hausdorff distance was 68.2+111.4 μm, and the median surface distance is 11.8±5.5 μm. The maximum MSD is 82.8 μm, and the minimum MSD is 6.9 μm. After applying the elastic registration, we find that the MSD is 13.6+12.2 μm, the 95% Hausdorff distance was 35.4+54.4 μm, and the median surface distance is 9.5+4.0 μm. The maximum MSD is 57.2 μm, and the minimum MSD is 6.4 μm. We observe that the MSD is less than the lateral pixel size of 30.15 μm but greater than the axial pixel size of 1.96 μm.

### C. Elastic Registration Decreases Montaging Errors

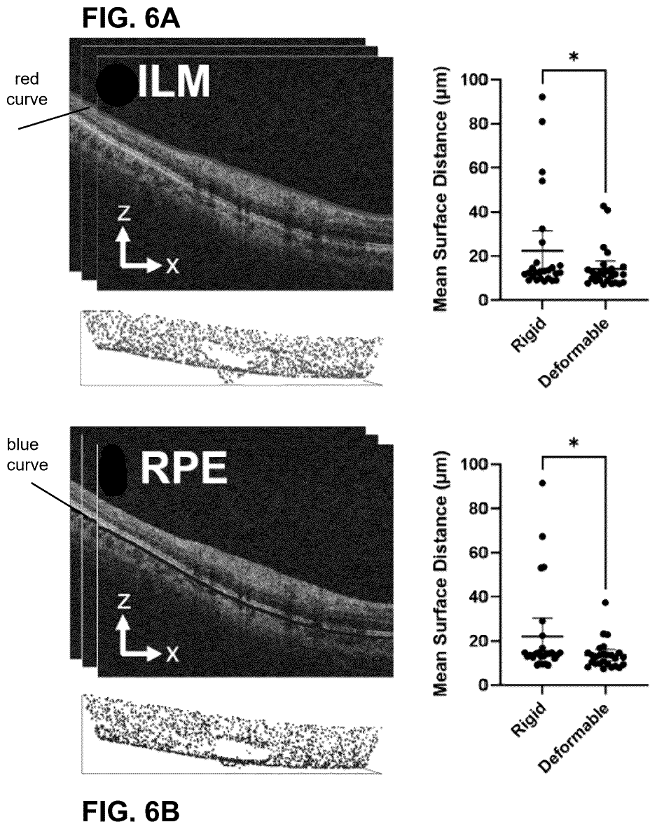

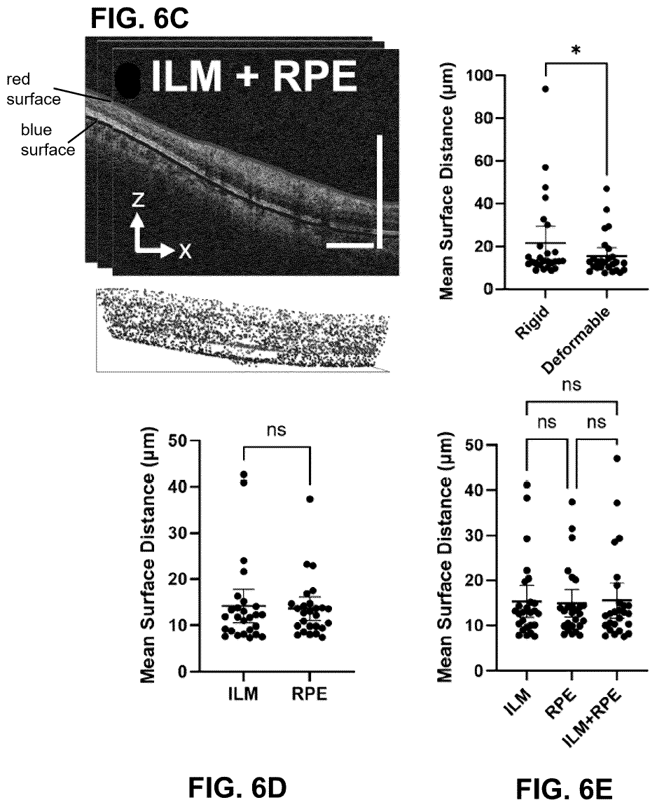

We compare the MSD after applying the rigid and the elastic transformations. FIG. 6A shows a B-scan image, with the ILM boundary highlighted in red. We show the point cloud corresponding to the ILM boundary below the B-scan. For the eyes (n=26 eyes) acquired from Wills Eye Hospital and Northwestern Memorial Hospital, which had similar single-volume FOV, we perform volumetric montaging using the ILM point clouds. We find that the MSD is smaller (p=0.02) after elastic registration (14.2±9.1 μm) than after rigid registration (22.4±22.8 μm).

FIG. 6B illustrates a B-scan image with the RPE boundary highlighted by the blue curve. Below the B-scan, we show the point cloud corresponding to the RPE boundary. For the same set of eyes, we performed volumetric montaging using the RPE point clouds. We found that the MSD was smaller (p=0.02) after elastic registration (13.6±6.4 μm) than after rigid registration (22.1±20.6 μm).

FIG. 6C shows a multi-layer point cloud generated using the ILM and RPE borders. The red surface depicts the topology of the ILM, and the blue surface depicts the topology of the RPE. We perform volumetric montaging using the multi-layer point clouds. We also found that the MSD is smaller (p=0.04) after elastic registration (15.5+9.7 μm) than after rigid registration (21.6±19.4 μm).

### D. Comparison of Point Cloud Generation Methodology

We evaluate whether generating the point cloud using the ILM, the RPE, or both affect montaging errors. We perform volumetric montaging using point clouds generated from the ILM and measured the MSD of the cumulative ILM point cloud. We repeat the same procedure but use the RPE instead of the ILM. We find that the cumulative ILM point cloud MSD is 14.2±9.1 μm (n=26 eyes) and that the cumulative RPE point cloud MSD is 13.6±6.4 μm (FIG. 6D), not statistically different than the ILM MSD (p=0.49).

We further investigate the impact of the point cloud generation method on the MSD for the multi-layer cumulative point cloud. We find no statistically significant difference between the methodologies for generating point clouds (FIG. 6E). The MSD for the cumulative multi-layer point cloud is 15.3±8.8 μm when the ILM point clouds are used to create the transformations, is 14.9+7.7 μm when the RPE point clouds are used, and 15.5+9.7 μm when the combined multi-layer point clouds are used.

In this example, we present a method for volumetrically montaging 3D OCT volumes acquired by three OCT devices (Spectralis2TM, Heidelberg Engineering; Cirrus™ HD-OCT, Zeiss; Maestro2™, Topcon). We demonstrate that 3D montaging of OCT volumes increases the FOV and axial depth without compromising high lateral sampling density. Most literature reports on OCT montaging and stitching are performed on 2D projection images. While scientists have learned much about the retina using stitched 2D projections, those methods fail to exploit the 3D nature of OCT fully. Other investigators have performed 3D registration ex vivo, primarily accounting for translation between volumes. We montage 3D OCT retinal volumes from human subjects. We represent the ILM and RPE of each volume as point clouds and apply point cloud registration to map the reference frames of each OCT volume into a common reference frame. By accounting for rotation, our method is robust to eye movement between acquired volumes, whose orientation to the incident OCT beam may differ substantially.

We test our method using datasets acquired at three institutions using different OCT machines and acquisition protocols. For data from all institutions, we successfully expand the FOV to more than 17 mm in the lateral directions. The increase FOV is comparable to that achievable with the latest ultra-widefield OCT, which has a FOV of around 20 mm in each lateral dimension [18, 19]. One benefit of 3D montaging is that it does not require new, expensive ultrawide OCT hardware. Since our methodology is not specific to any data-acquisition scheme, it can potentially be retroactively applied to historical data acquired from clinical OCT systems.

Two additional advantages of 3D montaging are denser sampling and an effective increase in axial imaging depth, which are usually constrained by either spectrometer design or the choice of a swept-source laser. For montaged datasets, the axial depth of the montaged volume is typically 1.5 to 2 times larger than that of a single OCT volume. We note that increasing axial depth helps minimize mirror artifacts caused by insufficient imaging depth in OCT volumes, allowing individual regions without mirror artifacts to be montaged together. This increased imaging depth is particularly valuable in eyes with an elongated axial length, such as high myopia.

We quantify the montaging error by computing the MSD between the point clouds of the registered volumes. We find that the average MSD between point clouds after registration is less than the pixel size in the lateral dimension across all institutions, suggesting that the montaging algorithm maintains the accuracy of anatomical structure location up to the lateral spatial sampling distance of OCT. Additionally, nearly all A-lines in the montaged volume had well-distinguishable retinal layers. If any volumes are misaligned, we would have observed a misalignment of retinal layers for some regions of the montaged volume. Instead, we find that the structures of all volumes in the montaged volume aligned well.

We find that the MSD of the cumulative point cloud comprising the ILM and RPE is not statistically different, regardless of whether the ILM, RPE, or both layers were used to compute rigid and elastic transformations. This suggests that segmenting single layers of the eye is a reasonable alternative to using both layers to generate the point.

Additional applications for our volumetric montaging include compensating for motion artifacts within individual scans [43, 44] by treating regions of the volume acquired before and after motion as different volumes to be registered. Additionally, the volumetric registration method can potentially average volumes acquired at the exact location to increase signal quality [45]. Moreover, volumetric registration can merge volumes of the same region acquired at different angles, potentially improving the lateral resolution of OCT and bringing it closer to the axial resolution [46-49]. Applications include measuring retinal curvature over a wider FOV, characterize the retinal topology in pathological myopia, and identifying the 3D structure of peripheral lesions. Overall, our 3D montaging method facilitate visualization of the retina's 3D structure across a larger FOV with enhanced understanding of how various disease states affect retinal structure.

### References for Example 1

[1] Huang, D., Swanson, E. A., Lin, C. P., Schuman, J. S., Stinson, W. G., Chang, W., Hee, M. R., Flotte, T., Gregory, K., Puliafito, C. A., and et al.: ‘Optical coherence tomography’, Science, 1991, 254, (5035), pp. 1178-1181.
[2] Jia, Y., Bailey, S. T., Hwang, T. S., McClintic, S. M., Gao, S. S., Pennesi, M. E., Flaxel, C. J., Lauer, A. K., Wilson, D. J., Hornegger, J., Fujimoto, J. G., and Huang, D.: ‘Quantitative optical coherence tomography angiography of vascular abnormalities in the living human eye’, Proc Natl Acad Sci USA, 2015, 112, (18), pp. E2395-2402.
[3] Varadarajan, A. V., Bavishi, P., Ruamviboonsuk, P., Chotcomwongse, P., Venugopalan, S., Narayanaswamy, A., Cuadros, J., Kanai, K., Bresnick, G., Tadarati, M., Silpa-archa, S., Limwattanayingyong, J., Nganthavee, V., Ledsam, J. R., Keane, P. A., Corrado, G. S., Peng, L., and Webster, D. R.: ‘Predicting optical coherence tomography-derived diabetic macular edema grades from fundus photographs using deep learning’, Nature Communications, 2020, 11, (1), pp. 130.
[4] Lee, S. W., Jeong, H. W., Kim, B. M., Ahn, Y. C., Jung, W., and Chen, Z.: ‘Optimization for Axial Resolution, Depth Range, and Sensitivity of Spectral Domain Optical Coherence Tomography at 1.3 μm’, J Korean Phys Soc, 2009, 55, (6), pp. 2354-2360.
[5] Duma, V.-F., Tankam, P., Huang, J., Won, J., and Rolland, J. P.: ‘Optimization of galvanometer scanning for optical coherence tomography’, Appl. Opt., 2015, 54, (17), pp. 5495-5507.
[6] Sadda, S. R., Keane, P. A., Ouyang, Y., Updike, J. F., and Walsh, A. C.: ‘Impact of scanning density on measurements from spectral domain optical coherence tomography’, Invest Ophthalmol Vis Sci, 2010, 51, (2), pp. 1071-1078.
[7] Barañano, A. E., Keane, P. A., Ruiz-Garcia, H., Walsh, A. C., and Sadda, S. R.: ‘Impact of scanning density on spectral domain optical coherence tomography assessments in neovascular age-related macular degeneration’, Acta Ophthalmologica, 2012, 90, (4), pp. e274-e280.
[8] Oncel, D., Manafi, N., nittala, M. G., Velga, S. B., Stambolian, D., Pericak-Vance, M. A., Haines, J. L., and Sadda, S. R.: ‘Effect of OCT B-scan Density on Sensitivity for Detection of Intraretinal Hyperreflective Foci’, Investigative Ophthalmology & Visual Science, 2022, 63, (7), pp. 4079—F0043-4079—F0043.
[9] Julia, W., Gregor, M., Henning, M., and Edmund, K.: ‘Signal power decrease due to fringe washout as an extension of the limited Doppler flow measurement range in spectral domain optical coherence tomography’, Journal of Biomedical Optics, 2010, 15, (4), pp. 041511.
[10] Ho, J., Castro, D. P., Castro, L. C., Chen, Y., Liu, J., Mattox, C., Krishnan, C., Fujimoto, J. G., Schuman, J. S., and Duker, J. S.: ‘Clinical assessment of mirror artifacts in spectral-domain optical coherence tomography’, Invest Ophthalmol Vis Sci, 2010, 51, (7), pp. 3714-3720.
[11] Zheng, S., Bai, Y., Xu, Z., Liu, P., and Ni, G.: ‘Optical Coherence Tomography for Three-Dimensional Imaging in the Biomedical Field: A Review’, Frontiers in Physics, 2021, 9.
[12] Saito, R., Shinohara, K., Tanaka, N., Takahashi, H., Yoshida, T., and Ohno-Matsui, K.: ‘ASSOCIATION BETWEEN DOME-SHAPED MACULA AND POSTERIOR STAPHYLOMA IN HIGHLY MYOPIC EYES INVESTIGATED BY ULTRA-WIDEFIELD OPTICAL COHERENCE TOMOGRAPHY’, Retina, 2021, 41, (3), pp. 646-652.
[13] Ludwig, C. A., Moon, J., Garg, I., and Miller, J. B.: ‘Ultra-Widefield Imaging for Evaluation of the Myopic Eye’, Semin Ophthalmol, 2021, 36, (4), pp. 185-190.
[14] Shinohara, K., Shimada, N., Moriyama, M., Yoshida, T., Jonas, J. B., Yoshimura, N., and Ohno-Matsui, K.: ‘Posterior Staphylomas in Pathologic Myopia Imaged by Widefield Optical Coherence Tomography’, Invest Ophthalmol Vis Sci, 2017, 58, (9), pp. 3750-3758.
[15] Bacherini, D., Rizzo, C., Vicini, G., Luciani, D., Vannozzi, L., Virgili, G., Giansanti, F., and Nicolosi, C.: ‘Characterization of Peripheral Retinal Degenerations and Rhegmatogenous Lesions Using Ultra-Widefield Swept Source OCT Integrated with a Novel Scanning Laser Ophthalmoscope’, Diagnostics (Basel), 2025, 15, (22).
[16] Wang, F., Saraf, S. S., Zhang, Q., Wang, R. K., and Rezaei, K. A.: ‘Ultra-Widefield Protocol Enhances Automated Classification of Diabetic Retinopathy Severity with OCT Angiography’, Ophthalmol Retina, 2020, 4, (4), pp. 415-424.
[17] Amato, A., Nadin, F., Borghesan, F., Cicinelli, M. V., Chatziralli, I., Sadiq, S., Mirza, R., and Bandello, F.: ‘Widefield Optical Coherence Tomography Angiography in Diabetic Retinopathy’, J Diabetes Res, 2020, 2020, pp. 8855709.
[18] Ripa, M., Motta, L., Florit, T., Sahyoun, J. Y., Matello, V., and Parolini, B.: ‘The Role of Widefield and Ultra Widefield Optical Coherence Tomography in the Diagnosis and Management of Vitreoretinal Diseases’, Diagnostics (Basel), 2022, 12, (9).
[19] Soecknick, F., Breher, K., Nafar, Z., Kubach, S., Straub, J., Wahl, S., and Ziemssen, F.: ‘The clinical evaluation of a widefield lens to expand the field of view in optical coherence tomography (OCT-A)’, Scientific Reports, 2024, 14, (1), pp. 6936.
[20] Ortiz, P., Draelos, M., Narawane, A., McNabb, R. P., Kuo, A. N., and Izatt, J. A.: ‘Robotically-Aligned Optical Coherence Tomography with Gaze Tracking for Live Image Montaging of the Retina’, in Editor (Ed.)\hat{ }(Eds.): ‘Book Robotically-Aligned Optical Coherence Tomography with Gaze Tracking for Live Image Montaging of the Retina’ (2022, edn.), pp. 3783-3789.
[21] Huang, Y., Li, X., Liu, J., Qiao, Z., Chen, J., and Hao, Q.: ‘Robotic-arm-assisted flexible large field-of-view optical coherence tomography’, Biomed. Opt. Express, 2021, 12, (7), pp. 4596-4609.
[22] Li, X., Huang, Y., and Hao, Q.: ‘Automated robot-assisted wide-field optical coherence tomography using structured light camera’, Biomed Opt Express, 2023, 14, (8), pp. 4310-4325.
[23] Fang, R., Zhang, P., Kim, D., Kweon, J., Sun, C., Huang, A. S., and Zhang, H. F.: ‘Robotic Visible-Light Optical Coherence Tomography Visualizes Segmental Schlemm's Canal Anatomy and Segmental Pilocarpine Response’, Invest Ophthalmol Vis Sci, 2025, 66, (2), pp. 47.
[24] Kim, D., Fang, R., Zhang, P., Yan, Z., Sun, C., Li, G., Montgomery, C., John, S. W. M., Stamer, W. D., Zhang, H. F., and Ethier, C. R.: ‘In Vivo Quantification of Anterior and Posterior Chamber Volumes in Mice: Implications for Aqueous Humor Dynamics’, Invest Ophthalmol Vis Sci, 2025, 66, (1), pp. 18.
[25] Mehta, N., Cheng, Y., Alibhai, A. Y., Duker, J. S., Wang, R. K., and Waheed, N. K.: ‘Optical coherence tomography angiography distortion correction in widefield montage images’, Quant Imaging Med Surg, 2021, 11, (3), pp. 928-938.
[26] Lewis, W., Kubach, S., Nafar, Z., and Pramil, V.: ‘Distortion correction of optical coherence tomography (OCT) scans for montage creation’, Investigative Ophthalmology & Visual Science, 2021, 62, (11), p. 48.
[27] De Sisternes, L., Lewis, W., Callan, T., Kubach, S., Bonnin, S., Tadayoni, R., and Durbin, M.: ‘OCTA volume combination for montaging and averaging’, Investigative Ophthalmology & Visual Science, 2020, 61, (9), pp. PB0086-PB0086.
[28] Schwarzhans, F., Desissaire, S., Steiner, S., Pircher, M., Hitzenberger, C. K., Resch, H., Vass, C., and Fischer, G.: ‘Generating large field of view en-face projection images from intra-acquisition motion compensated volumetric optical coherence tomography data’, Biomed Opt Express, 2020, 11, (12), pp. 6881-6904.
[29] Gan, Y., Yao, W., Myers, K. M., and Hendon, C. P.: ‘An automated 3D registration method for optical coherence tomography volumes’, Annu Int Conf IEEE Eng Med Biol Soc, 2014, 2014, pp. 3873-3876.
[30] Laves, M.-H., Kahrs, L. A., and Ortmaier, T.: ‘Volumetric 3D stitching of optical coherence tomography volumes’, 2018, 4, (1), pp. 327-330.
[31] Li, Y., Gregori, G., Lam, B. L., and Rosenfeld, P. J.: ‘Automatic montage of SD-OCT data sets’, Opt Express, 2011, 19, (27), pp. 26239-26248.
[32] Mori, K., Kanno, J., and Gehlbach, P. L.: ‘RETINOCHOROIDAL MORPHOLOGY DESCRIBED BY WIDE-FIELD MONTAGE IMAGING OF SPECTRAL DOMAIN OPTICAL COHERENCE TOMOGRAPHY’, Retina, 2016, 36, (2), pp. 375-384.
[33] Ji, Y., Zhou, K., Ibbotson, S. H., Wang, R. K., Li, C., and Huang, Z.: ‘A novel automatic 3D stitching algorithm for optical coherence tomography angiography and its application in dermatology’, Journal of Biophotonics, 2021, 14, (11), pp. e202100152.
[34] https://github.com/beasygoing/OCT-Retinal-Layer-Segmenter?tab=readme-ov-file.
[35] Li, L., Wang, R., and Zhang, X.: ‘A Tutorial Review on Point Cloud Registrations: Principle, Classification, Comparison, and Technology Challenges’, Mathematical Problems in Engineering, 2021, 2021, pp. 9953910.
[36] Blinn, J.: ‘Jim Blinn's Corner: A Trip Down the Graphics Pipeline’ (Morgan Kaufmann, 1996.).
[37] Lewis, J. P.: ‘Fast normalized cross-correlation’, in Editor (Ed.)\hat{ }(Eds.): ‘Book Fast normalized cross-correlation’ (1995, edn.), pp. 120-123.
[38] Pleansamai, K., and Chaiyasarn, K.: ‘M-estimator sample consensus planar extraction from image-based 3D point cloud for building information modelling’, GEOMATE Journal, 2019, 17, (63), pp. 69-76.
[39] Besl, P. J., and Mckay, N. D.: ‘Method for registration of 3-D shapes’, in Editor (Ed.)\hat{ }(Eds.): ‘Book Method for registration of 3-D shapes’ (Spie, 1992, edn.), pp. 586-606.
[40] Akkiraju, N., Edelsbrunner, H., Facello, M., Fu, P., Mucke, E., and Varela, C.: ‘Alpha shapes: definition and software’, in Editor (Ed.)\hat{ }(Eds.): ‘Book Alpha shapes: definition and software’ (1995, edn.).
[41] Zhang, M., Gofas-Salas, E., Leonard, B. T., Rui, Y., Snyder, V. C., Reecher, H. M., Mecê, P., and Rossi, E. A.: ‘Strip-based digital image registration for distortion minimization and robust eye motion measurement from scanned ophthalmic imaging systems’, Biomed Opt Express, 2021, 12, (4), pp. 2353-2372.
[42] Jonnal, R. S., Kocaoglu, O. P., Zawadzki, R. J., Liu, Z., Miller, D. T., and Werner, J. S.: ‘A Review of Adaptive Optics Optical Coherence Tomography: Technical Advances, Scientific Applications, and the Future’, Investigative Ophthalmology & Visual Science, 2016, 57, (9), pp. OCT51-OCT68.
[43] Lezama, J., Mukherjee, D., McNabb, R. P., Sapiro, G., Kuo, A. N., and Farsiu, S.: ‘Segmentation guided registration of wide field-of-view retinal optical coherence tomography volumes’, Biomed Opt Express, 2016, 7, (12), pp. 4827-4846.
[44] Sánchez Brea, L., Andrade De Jesus, D., Shirazi, M. F., Pircher, M., van Walsum, T., and Klein, S.: ‘Review on Retrospective Procedures to Correct Retinal Motion Artefacts in OCT Imaging’, in Editor (Ed.)\hat{ }(Eds.): ‘Book Review on Retrospective Procedures to Correct Retinal Motion Artefacts in OCT Imaging’ (2019, edn.).
[45] Hendargo, H. C., Estrada, R., Chiu, S. J., Tomasi, C., Farsiu, S., and Izatt, J. A.: ‘Automated non-rigid registration and mosaicing for robust imaging of distinct retinal capillary beds using speckle variance optical coherence tomography’, Biomed. Opt. Express, 2013, 4, (6), pp. 803-821.
[46] Bosch, C. M., Baumann, C., Dehghani, S., Sommersperger, M., Johannigmann-Malek, N., Kirchmair, K., Maier, M., and Nasseri, M. A.: ‘A Tool for High-Resolution Volumetric Optical Coherence Tomography by Compounding Radial- and Linear Acquired B-Scans Using Registration’, Sensors (Basel), 2022, 22, (3).
[47] Zhou, K. C., Qian, R., Degan, S., Farsiu, S., and Izatt, J. A.: ‘Optical coherence refraction tomography’, Nat Photonics, 2019, 13, (11), pp. 794-802.
[48] Chen, Y., Manzanera, S., Mompeán, J., Ruminski, D., Grulkowski, I., and Artal, P.: ‘Increased crystalline lens coverage in optical coherence tomography with oblique scanning and volume stitching’, Biomed Opt Express, 2021, 12, (3), pp. 1529-1542.
[49] Cheng, Y., Chu, Z., and Wang, R. K.: ‘Robust three-dimensional registration on optical coherence tomography angiography for speckle reduction and visualization’, Quant Imaging Med Surg, 2021, 11, (3), pp. 879-894.

### STATEMENTS REGARDING INCORPORATION BY REFERENCE AND VARIATIONS

All references throughout this application, for example patent documents including issued or granted patents or equivalents; patent application publications; and non-patent literature documents or other source material; are hereby incorporated by reference herein in their entireties, as though individually incorporated by reference, to the extent each reference is at least partially not inconsistent with the disclosure in this application (for example, a reference that is partially inconsistent is incorporated by reference except for the partially inconsistent portion of the reference).

The terms and expressions which have been employed herein are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments, exemplary embodiments and optional features, modification and variation of the concepts herein disclosed may be resorted to by those skilled in the art, and that such modifications and variations are considered to be within the scope of this invention as defined by the appended claims. The specific embodiments provided herein are examples of useful embodiments of the present invention and it will be apparent to one skilled in the art that the present invention may be carried out using a large number of variations of the devices, device components, methods steps set forth in the present description. As will be obvious to one of skill in the art, methods and devices useful for the present methods can include a large number of optional composition and processing elements and steps.

As used herein and in the appended claims, the singular forms “a”, “an”, and “the” include plural reference unless the context clearly dictates otherwise. Thus, for example, reference to “a cell” includes a plurality of such cells and equivalents thereof known to those skilled in the art. As well, the terms “a” (or “an”), “one or more” and “at least one” can be used interchangeably herein. It is also to be noted that the terms “comprising”, “including”, and “having” can be used interchangeably. The expression “of any of claims XX-YY” (wherein XX and YY refer to claim numbers) is intended to provide a multiple dependent claim in the alternative form, and in some embodiments is interchangeable with the expression “as in any one of claims XX-YY.”

When a group of substituents is disclosed herein, it is understood that all individual members of that group and all subgroups, are disclosed separately. When a Markush group or other grouping is used herein, all individual members of the group and all combinations and subcombinations possible of the group are intended to be individually included in the disclosure.

Whenever a range is given in the specification, for example, a temperature range, a time range, or a composition or concentration range, all intermediate ranges and subranges, as well as all individual values included in the ranges given are intended to be included in the disclosure. It will be understood that any subranges or individual values in a range or subrange that are included in the description herein can be excluded from the claims herein.

All patents and publications mentioned in the specification are indicative of the levels of skill of those skilled in the art to which the invention pertains. References cited herein are incorporated by reference herein in their entirety to indicate the state of the art as of their publication or filing date and it is intended that this information can be employed herein, if needed, to exclude specific embodiments that are in the prior art. For example, when composition of matter are claimed, it should be understood that compounds known and available in the art prior to Applicant's invention, including compounds for which an enabling disclosure is provided in the references cited herein, are not intended to be included in the composition of matter claims herein.

As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim. In each instance herein any of the terms “comprising”, “consisting essentially of” and “consisting of” may be replaced with either of the other two terms. The invention illustratively described herein suitably may be practiced in the absence of any element or elements, limitation or limitations which is not specifically disclosed herein.

One of ordinary skill in the art will appreciate that components and methods other than those specifically exemplified can be employed in the practice of the invention without resort to undue experimentation. All art-known functional equivalents, of any such components and methods are intended to be included in this invention. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

### REFERENCES (ADDITIONAL)

P. Ortiz, M. Draelos, A. Narawane, R. P. McNabb, A. N. Kuo and J. A. Izatt, “Robotically-Aligned Optical Coherence Tomography with Gaze Tracking for Live Image Montaging of the Retina,” 2022 International Conference on Robotics and Automation (ICRA), Philadelphia, PA, USA, 2022, pp. 3783-3789, doi: 10.1109/ICRA46639.2022.9812428.
Ripa, M., Motta, L., Florit, T., Sahyoun, J. Y., Matello, V., and Parolini, B.: ‘The Role of Wide field and Ultra Widefield Optical Coherence Tomography in the Diagnosis and Management of Vitreoretinal Diseases’, Diagnostics (Basel), 2022, 12, (9).
Ji, Y., Zhou, K., Ibbotson, S. H., Wang, R. K., Li, C., and Huang, Z.: ‘A novel automatic 3D stitching algorithm for optical coherence tomography angiography and its application in dermatology’, Journal of Biophotonics, 2021, 14, (11), pp. e202100152.
Li, Y., Gregori, G., Lam, B. L., and Rosenfeld, P. J.: ‘Automatic montage of SD-OCT data sets’, Opt Express, 2011, 19, (27), pp. 26239-26248
Laves, M.-H., Kahrs, L. A., and Ortmaier, T.: ‘Volumetric 3D stitching of optical coherence tomography volumes’, 2018, 4, (1), pp. 327-330:
Gan, Y., Yao, W., Myers, K. M., and Hendon, C. P.: ‘An automated 3D registration method for optical coherence tomography volumes’, Annu Int Conf IEEE Eng Med Biol Soc, 201 4, 2014, pp. 3873-3876.

We claim:

1. A method for expanding a field of view (FOV) in three dimensions of an object to be imaged by optical coherence tomography (OCT), the method comprising the steps of:
providing a plurality of OCT volumetric images comprising multiple overlapping pairs of OCT volumetric images;
representing each of the OCT volumetric images as a point cloud corresponding to a surface of each of the OCT volumetric images;
identifying common landmark point(s) between overlapping OCT volumetric images; mapping each of the OCT volumetric images to a common reference frame by:
a rigid transformation including translation, rotation and reflection of the common landmark point(s) between each of the overlapping pairs of OCT volumetric images;
refining the rigid transformation by recalculating the rigid transformation based on the point clouds of the overlapping region in addition to that of the landmark points;
a deformation registration to account for local deformations outside of the translation, rotation, and reflection of the common landmark point(s);
forming a merged volume from the mapped OCT volumetric images in the common reference frame; thereby expanding the FOV of the object.

2. The method of claim 1, wherein the mapping further comprises:
dividing the point cloud representing each of the OCT volumetric images into strips;
calculating a deformation vector field from the strips by a strip-based algorithm; and
using both the rigid transformation and the deformation vector field to map each of the OCT volumetric images to the common reference frame.

3. The method of claim 2, wherein the deformation vector field improves alignment of the mapped OCT volumetric images that are otherwise subject to motion-based errors.

4. The method of claim 1, wherein the plurality of OCT volumetric images comprises: actively obtaining the plurality of OCT volumetric images of the object or obtaining from a previously imaged object.

5. The method of claim 1, wherein a top surface of each of the OCT volumetric images is an internal limiting membrane (ILM).

6. The method of claim 1, wherein a top surface of a subset of the plurality of OCT volumetric images is a retinal anatomical layer after segmentation.

7. The method of claim 1, wherein the object is a retina.

8. The method of claim 7, wherein the merged volume is used to assess a surface topology parameter of the retina.

9. The method of claim 7, wherein the merged volume is used for one or more of: measuring a retinal curvature; diagnosing pathological myopia from retinal topology; characterizing a peripheral lesion (including 3D structure); point cloud-based eye tracking; or locating posterior staphylomas in a peripheral eye or retina region.

10. The method of claim 1, further comprising increasing a FOV and axial depth without adversely impacting a lateral sampling density.

11. The method of claim 1, wherein the expanding the FOV is characterized by a lateral scanning dimension that is greater than or equal to 18 mm.

12. The method of claim 1, wherein the merged volume has an increased axial depth compared to the provided OCT volumetric images, including 1.5 to 2.5 times an axial depth of an individual OCT volumetric image, thereby minimizing risk of a mirror artifact.

13. The method of claim 1, wherein a mean surface distance between adjacent point clouds after mapping is less than a pixel lateral dimension.

14. The method of claim 1, wherein the expanding the FOV is characterized by:
an increase in a lateral surface area between an individual OCT volumetric image lateral surface area (e.g., 30 mm² to 100 mm²) and the merged volume lateral surface area (100 mm² to 350 mm²), including a ratio of between 2.5 and 4.0;
an increase in a volume between an individual OCT volumetric image volume (e.g., 70 mm³ to 175 mm³) and the merged volume (e.g., 250 mm³ to 1,100 mm³), including a ratio of between 3.5 and 7.

15. A system for constructing an expanded FOV of an object to be imaged from a plurality of OCT volumetric images, the system comprising:
an OCT imaging system; and
processing circuitry configured to:
construct, based on data provided by the OCT imaging system, a plurality of OCT volumetric images; and
perform the method according to claim 1.

16. A non-transitory computer readable medium having stored thereon processor executable instructions that, when executed by one or more processors, cause the one or more processors to perform the method according to claim 1.
